(19) United States
(12) Patent Application Publication
(10) Pub. No.: US 2007/0243152 A1
(43) Pub. Date: Oct. 18, 2007

(54) Formula for fabricating skin treatment product and forming method thereof

(76) Inventors: Chien-Chang Wang, Taipei (TW); Chao-An Chen, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/403,856

(22) Filed: Apr. 14, 2006

(51) Int. Cl. A61K 8/00

(52) U.S. Cl. 424/70.1

(57) ABSTRACT

The present invention discloses a formula for fabricating skin treatment product, wherein the formula comprises at least one pectin enzyme and at least one cellulose enzyme. The formula is used to mix with an organic mixture to fabricate the desired skin treatment product, and the organic mixture comprises extract or artifact of nature product. Additionally, this invention also discloses a method for fabricating skin treatment product by use of the above-mentioned formula.

### BACKGROUND OF THE INVENTION

1. Field of the Invention

The present invention is generally related to a formula for fabricating skin treatment product and the fabrication method thereof, and more particularly, a formula comprising enzymes for fabricating skin treatment product.

2. Description of the Prior Art

Homemade skin treatment product becomes very popular throughout the world. Thus, people often purchase the raw materials from chemical stores to fabricate simple skin treatment product. However, after applying such homemade facial mask or skin treatment product, symptoms of facial red and swollen, spots, or contact dermatitis are shown on the face. Natural fresh produce may have plenty of nutrition for human skin and can be used to fabricate skin product but the natural fresh produce includes some ingredients, such as pectin and coarse fiber, which are not or are hard to be absorbed directly by skin to have the effect of nourishment. Therefore, it is an important research topic to promote the absorbability of the skin treatment product made by nature fresh produce and to provide a simpler method for fabricating treatment product.

### SUMMARY OF THE INVENTION

According to the above, the present invention provides a formula for fabricating skin treatment product and a method for fabricating the same to fulfill the requirements of this industry.

One object of the present invention is to provide a formula comprising an enzyme for fabricating skin treatment product. Because of the effects of decomposition and transformation by the enzymes in the formula, some ingredients in natural fresh produce, such as pectin and coarse fiber, which are not or are hard to be absorbed directly by skin can be decomposed so that human skin can absorb the nutrition in the natural fresh produce to achieve skin whitening and smoothing effect.

Another object of the present invention is to provide a formula comprising at least two enzymes for fabricating skin treatment product. Synergistic effect of composite enzymes is more effective than single enzyme. Furthermore, enzyme itself can activate skin cells, promote skin cells to regenerate, and decompose melanin. Therefore, the present invention meets the requirements of the industry.

According to the described objects, the invention provides a formula for fabricating skin treatment product comprising at least one pectin enzyme and at least one cellulose enzyme. Besides, the formula is used to mix with an organic mixture to fabricate the desired skin treatment product. The organic mixture comprises extract or artifact of nature product. On the other hand, the invention provides a method for fabricating skin treatment product.

### DESCRIPTION OF THE PREFERRED EMBODIMENTS

What is probed into the invention is a formula for fabricating skin treatment product. Detail descriptions of the processes and compositions will be provided in the following in order to make the invention thoroughly understood. The invention uses the percentage by weight to express the amount of each composition in the formula since the weight percentage is most commonly used by those who are skilled in the art. Obviously, the application of the invention is not confined to specific details familiar to those who are skilled in the art. On the other hand, the common processes and compositions that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

The first embodiment of the invention discloses a formula for fabricating skin treatment product comprising at least one pectin enzyme and at least one cellulose enzyme. The formula is used to mix with an organic mixture to fabricate the desired skin treatment product. Moreover, the organic mixture comprises extract or artifact of nature product and the nature product includes pectin and/or cellulose. The organic mixture includes one selected from a group consisting of the following: vegetable juice, fruit juice, tea, milk, alcohol, and honey.

In the embodiment, the pectin enzyme includes one selected from a group consisting of the following: pectinase, pectinesterase, polygalacturonase, and pectin lyase while the cellulose enzyme includes one selected from a group consisting of the following: endo-1.4-β-D-glucanase, exo-1.4-β-D-glucanase, and β-1.4-glucosidase. Besides, the ratio of said pectin enzyme to said cellulose enzyme by weight ranges from 1 to 10 and the preferred value is about 4. In addition, the amount of the pectin enzyme and the cellulose enzyme is about 40%˜90% of the total weight of the formula and the preferred value is about 60% by weight in which the activation unit of the pectin enzyme is about 3000 and that of the cellulose enzyme is about 7200. In a preferred embodiment of this embodiment, the formula further comprises pashanbheda extract SK in an amount of about 1 wt % of the formula. In another preferred embodiment of this embodiment, the formula further comprises oat beta-glucan in an amount of about 1 wt % of the formula. In another preferred embodiment of this embodiment, the formula further comprises crtyl ethylhexanoate in an amount of about 1 wt % of the formula.

### EXAMPLE 1

A formula for fabricating skin treatment product, that is suitable to be used with an organic mixture with one time of the volume of the formula, comprises: (A) 3.8˜4.2 wt % of 1,3-butylene glycol, 0.19˜0.21 wt % of methyl p-hydroxybenzoate, 0.855˜0.945 wt % of the solution with 1.9˜2.1 wt % hydroxytehyl cellulose, 57˜63 wt % of pectin enzyme and cellulose enzyme, 14.06˜15.54 wt % of deionized water, and 0.57˜0.63 wt % of D-pantenol; (B) 0.19˜0.21 wt % of isopropylmethylphenol, 9.5˜10.5 wt % of PEG-60 hydrogenated castor oil, 0.095˜0.105 wt % of dipotassium glycyrrhizinate, 9.5˜10.5 wt % of the solution with 0.95˜1.05 wt % sodium hyaluronate, 0.95˜1.05 wt % of mulberry bark extract, and 0.95˜1.05 wt % of soy extract; and, (C) 0.95˜1.05 wt % of pashanbheda extract SK, 0.95˜1.05 wt % of rose apple leaf extract, 0.95˜1.05 wt % of oat beta-glucan, and 0.95˜1.05 wt % of crtyl ethylhexanoate.

### EXAMPLE 2

A formula for fabricating skin treatment product, that is suitable to be used with an organic mixture with seven times of the volume of the formula, comprises: (A) 3.8˜4.2 wt % of 1,3-butylene glycol, 1.425˜1.575 wt % of methyl p-hydroxybenzoate, 0.855˜0.945 wt % of the solution with 1.9˜2.1 wt % hydroxytehyl cellulose, 57˜63 wt % of pectin enzyme and cellulose enzyme, 14.06˜15.54 wt % of deionized water, and 0.57˜0.63 wt % of D-pantenol; (B) 0.19˜0.21 wt % of isopropylmethylphenol, 9.5˜10.5 wt % of PEG-60 hydrogenated castor oil, 0.095˜0.105 wt % of dipotassium glycyrrhizinate, 8.36˜9.24 wt % of the solution with 0.95˜1.05 wt % sodium hyaluronate, 0.95˜1.05 wt % of mulberry bark extract, and 0.95˜1.05 wt % of soy extract; and, (C) 1.425˜1.575 wt % of pashanbheda extract SK, 1.425˜1.575 wt % of rose apple leaf extract, 0.95˜1.05 wt % of oat beta-glucan, and 0.95˜1.05 wt % of crtyl ethylhexanoate.

The second embodiment of the invention discloses a method for fabricating skin treatment product. At first, an organic mixture is provided. The organic mixture comprises extract or artifact of nature product that includes pectin and/or cellulose. Then, a formula for fabricating skin treatment product is provided. The formula comprises at least one pectin enzyme and at least one cellulose enzyme. By thoroughly mixing the organic mixture with the formula, the general purpose skin treatment product is fabricated. The mixing ratio of the organic mixture to the formula by volume ranges from 0.5 to 12.

In the embodiment, the selections for the above described organic mixture, pectin enzyme, cellulose enzyme, the ratio of the pectin enzyme to the cellulose enzyme, the total amount of the pectin enzyme and the cellulose enzyme, the preferred formula for fabricating skin treatment product, and the amount of each composition in the formula are in the same manner as those in the first embodiment. In a preferred embodiment of this embodiment, the formula further comprises pashanbheda extract SK in an amount of about 1 wt % of the formula so as to prevent skins from aging and increase elasticity of skins. In another preferred embodiment of this embodiment, the formula further comprises oat beta-glucan in an amount of about 1 wt % of the formula. In another preferred embodiment of this embodiment, the formula further comprises crtyl ethylhexanoate in an amount of about 1 wt % of the formula so as to have capability of moisture retention.

Fabrication Processes for General Purpose Skin Treatment Product

The fabrication processes include the steps of squeezing juice out of fresh vegetables, such as cucumber, or fresh fruits, such as lemon, orange, and kiwi fruit, subsequently using non-woven fabric to filter coarse residue out of the juice, and finally mixing the processed juice with the formula according to the invention with specific ratio thoroughly to complete the skin treatment product. The mixing process needs powerful agitation in order to have the mixture uniform. In addition, the skin treatment product can be diluted to three times of volume before use, depending on personal preference.

Recommendation of Use for General Purpose Skin Treatment Product

The usage of skin treatment product includes taking small amount of the skin treatment product to apply on the skin and then using hands to soak in water to thereby apply on the skin for 3˜5 times. The enzymes need the environment with plenty of water in order to function. According to the experimental verification, pectin enzymes can remove pectin in the juice and cellulose enzyme can decompose fibers in the juice to generate glucose, referred to Schemes 1 and 2, so as to let the nutrition be transformed to small molecules to be absorbable by human skins.

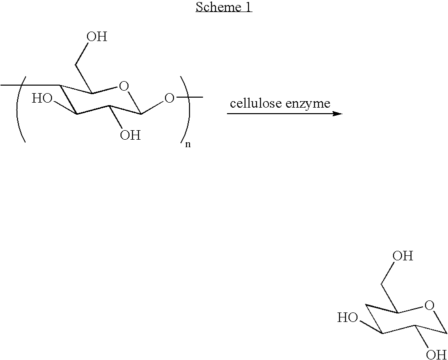

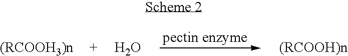

To sump up, the present invention discloses a formula for fabricating skin treatment product, wherein the formula comprises at least one pectin enzyme and at least one cellulose enzyme. The formula is used to mix with an organic mixture to fabricate the desired skin treatment product. The organic mixture comprises extract or artifact of nature product. Additionally, this invention also discloses a method for fabricating skin treatment product.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A formula for fabricating skin treatment product, comprising:
at least one pectin enzyme, wherein said pectin enzyme includes one selected from a group consisting of the following: pectinase, pectinesterase, polygalacturonase, and pectin lyase; and,
at least one cellulose enzyme, wherein said cellulose enzyme includes one selected from a group consisting of the following: endo-1.4-β-D-glucanase, exo-1.4-β-D-glucanase, and β-1.4-glucosidase;
wherein said formula is used to mix with an organic mixture to fabricate the desired skin treatment product, said organic mixture comprises extract or artifact of nature product, and said nature product includes pectin and/or cellulose.

2. The formula for fabricating skin treatment product according to claim 1, wherein the ratio of said pectin enzyme to said cellulose enzyme by weight ranges from 1 to 10.

3. The formula for fabricating skin treatment product according to claim 1, wherein the ratio of said pectin enzyme to said cellulose enzyme by weight is about 4.

4. The formula for fabricating skin treatment product according to claim 1, wherein the amount of said pectin enzyme and said cellulose enzyme is about 40%˜90% of the total weight of said formula.

5. The formula for fabricating skin treatment product according to claim 1, wherein the amount of said pectin enzyme and said cellulose enzyme is about 60% of the total weight of said formula.

6. The formula for fabricating skin treatment product according to claim 1, wherein said organic mixture includes one selected from a group consisting of the following: vegetable juice, fruit juice, tea, milk, alcohol, and honey.

7. The formula for fabricating skin treatment product according to claim 1, wherein said formula comprises:
(A) 3.8˜4.2 wt % of 1,3-butylene glycol, 0.19˜0.21 wt % of methyl p-hydroxybenzoate, 0.855˜0.945 wt % of the solution with 1.9˜2.1 wt % hydroxytehyl cellulose, 57˜63 wt % of pectin enzyme and cellulose enzyme, 14.06˜15.54 wt % of deionized water, and 0.57˜0.63 wt % of D-pantenol;
(B) 0.19˜0.21 wt % of isopropylmethylphenol, 9.5˜10.5 wt % of PEG-60 hydrogenated castor oil, 0.095˜0.105 wt % of dipotassium glycyrrhizinate, 9.5˜10.5 wt % of the solution with 0.95˜1.05 wt % sodium hyaluronate, 0.95˜1.05 wt % of mulberry bark extract, and 0.95˜1.05 wt % of soy extract; and,
(C) 0.95˜1.05 wt % of pashanbheda extract SK, 0.95˜1.05 wt % of rose apple leaf extract, 0.95˜1.05 wt % of oat beta-glucan, and 0.95˜1.05 wt % of crtyl ethylhexanoate.

8. The formula for fabricating skin treatment product according to claim 1, wherein said formula comprises:
(A) 3.8˜4.2 wt % of 1,3-butylene glycol, 1.425˜1.575 wt % of methyl p-hydroxybenzoate, 0.855˜0.945 wt % of the solution with 1.9˜2.1 wt % hydroxytehyl cellulose, 57˜63 wt % of pectin enzyme and cellulose enzyme, 14.06˜15.54 wt % of deionized water, and 0.57˜0.63 wt % of D-pantenol;
(B) 0.19˜0.21 wt % of isopropylmethylphenol, 9.5˜10.5 wt % of PEG-60 hydrogenated castor oil, 0.095˜0.105 wt % of dipotassium glycyrrhizinate, 8.36˜9.24 wt % of the solution with 0.95˜1.05 wt % sodium hyaluronate, 0.95˜1.05 wt % of mulberry bark extract, and 0.95˜1.05 wt % of soy extract; and,
(C) 1.425˜1.575 wt % of pashanbheda extract SK, 1.425˜1.575 wt % of rose apple leaf extract, 0.95˜1.05 wt % of oat beta-glucan, and 0.95˜1.05 wt % of crtyl ethylhexanoate.

9. A method for fabricating skin treatment product, comprising:
providing an organic mixture comprising extract or artifact of nature product wherein said nature product includes pectin and/or cellulose;
providing a formula for fabricating skin treatment product including at least one pectin enzyme and at lease one cellulose enzyme, wherein said pectin enzyme includes one selected from a group consisting of the following: pectinase, pectinesterase, polygalacturonase, and pectin lyase, said cellulose enzyme includes one selected from a group consisting of the following: endo-1.4-β-D-glucanase, exo-1.4-β-D-glucanase, and β-1.4-glucosidase; and,
mixing said formula for fabricating skin treatment product with said organic mixture so as to form said skin treatment product.

10. The method for fabricating skin treatment product according to claim 9, wherein said organic mixture includes one selected from a group consisting of the following: vegetable juice, fruit juice, tea, milk, alcohol, and honey.

11. The method for fabricating skin treatment product according to claim 9, wherein the ratio of said pectin enzyme to said cellulose enzyme by weight ranges from 1 to 10.

12. The method for fabricating skin treatment product according to claim 9, wherein the ratio of said pectin enzyme to said cellulose enzyme by weight is about 4.

13. The method for fabricating skin treatment product according to claim 9, wherein the amount of said pectin enzyme and said cellulose enzyme is about 40%˜90% of the total weight of said formula.

14. The method for fabricating skin treatment product according to claim 17, wherein the mixing ratio of said organic mixture to said formula by volume ranges from 0.5 to 12.

15. The method for fabricating skin treatment product according to claim 9, wherein said formula comprises:
(A) 3.8˜4.2 wt % of 1,3-butylene glycol, 0.19˜0.21 wt % of methyl p-hydroxybenzoate, 0.855˜0.945 wt % of the solution with 1.9˜2.1 wt % hydroxytehyl cellulose, 57˜63 wt % of pectin enzyme and cellulose enzyme, 14.06˜15.54 wt % of deionized water, and 0.57˜0.63 wt % of D-pantenol;
(B) 0.19˜0.21 wt % of isopropylmethylphenol, 9.5˜10.5 wt % of PEG-60 hydrogenated castor oil, 0.095˜0.105 wt % of dipotassium glycyrrhizinate, 9.5˜10.5 wt % of the solution with 0.95˜1.05 wt % sodium hyaluronate, 0.95˜1.05 wt % of mulberry bark extract, and 0.95˜1.05 wt % of soy extract; and,
(C) 0.95˜1.05 wt % of pashanbheda extract SK, 0.95˜1.05 wt % of rose apple leaf extract, 0.95˜1.05 wt % of oat beta-glucan, and 0.95˜1.05 wt % of crtyl ethylhexanoate.

16. The method for fabricating skin treatment product according to claim 9, wherein said formula comprises:
(A) 3.8˜4.2 wt % of 1,3-butylene glycol, 1.425˜1.575 wt % of methyl p-hydroxybenzoate, 0.855˜0.945 wt % of the solution with 1.9˜2.1 wt % hydroxytehyl cellulose, 57˜63 wt % of pectin enzyme and cellulose enzyme, 14.06˜15.54 wt % of deionized water, and 0.57˜0.63 wt % of D-pantenol;
(B) 0.19˜0.21 wt % of isopropylmethylphenol, 9.5˜10.5 wt % of PEG-60 hydrogenated castor oil, 0.095˜0.105 wt % of dipotassium glycyrrhizinate, 8.36˜9.24 wt % of the solution with 0.95˜1.05 wt % sodium hyaluronate, 0.95˜1.05 wt % of mulberry bark extract, and 0.95˜1.05 wt % of soy extract; and,
(C) 1.425˜1.575 wt % of pashanbheda extract SK, 1.425˜1.575 wt % of rose apple leaf extract, 0.95˜1.05 wt % of oat beta-glucan, and 0.95˜1.05 wt % of crtyl ethylhexanoate.
